(19) United States
(12) Patent Application Publication
(10) Pub. No.: US 2026/0051409 A1
(43) Pub. Date: Feb. 19, 2026

(54) SYSTEMS AND METHODS FOR PREDICTING INCIDENT ADENOCARCINOMA OF THE ESOPHAGUS OR GASTRIC CARDIA USING MACHINE LEARNING

(71) Applicant: REGENTS OF THE UNIVERSITY OF MICHIGAN, Ann Arbor, MI (US)

(72) Inventors: Ji Zhu, Ann Arbor, MI (US); Akbar Waljee, Ann Arbor, MI (US); Joel Rubenstein, Ann Arbor, MI (US)

(21) Appl. No.: 19/299,960

(22) Filed: Aug. 14, 2025

Related U.S. Application Data
(60) Provisional application No. 63/682,912, filed on Aug. 14, 2024.

(51) Int. Cl. G16H 50/30; G06N 20/00; G16H 10/60; G16H 20/00; G16H 50/70

(52) U.S. Cl. CPC G16H 50/30; G06N 20/00; G16H 10/60; G16H 20/00; G16H 50/70

(57) ABSTRACT

Systems and methods for predicting esophageal adenocarcinoma (EAC) and gastric cardia adenocarcinoma (GCA) using machine learning are provided. An example system may obtain an electronic health record (EHR) dataset, identify missing values in the EHR dataset, and generate imputed values for the missing values using simple random sampling imputation. The system may train a model using an extreme gradient boosting algorithm and a training dataset including the EHR dataset to generate a trained model including multiple decision trees. Training the model includes tuning the model to achieve a greatest value of an area under a receiver operating characteristic curve associated with the model. The system may obtain a patient EHR dataset, generate a prediction associated with a risk of EAC and/or GCA by applying the trained model to the patient EHR dataset, and provide the prediction to a computing device to determine a patient treatment protocol.

### CROSS REFERENCE TO RELATED APPLICATIONS

This application claims the benefit of U.S. Provisional Application No. 63/682,912, entitled “Systems And Methods For Predicting Incident Adenocarcinoma Of The Esophagus Or Gastric Cardia Using Machine Learning” (filed Aug. 14, 2024), the entirety of which is incorporated by reference herein.

### GOVERNMENT LICENSE RIGHTS

This invention was made with government support under W81XWH-20-1-0898 awarded by the U.S. Defense Health Agency, Medical Research and Development Branch. The government has certain rights in the invention.

### FIELD OF THE DISCLOSURE

The present disclosure generally relates to systems and methods for predicting cancer, and more particularly, to systems and methods for predicting incident adenocarcinoma of the esophagus or gastric cardia using machine learning.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Incidences of esophageal adenocarcinoma (EAC) have risen dramatically over the past few decades. If precursor lesions in the esophagus are detected early enough in at-risk patients via esophageal cancer screenings, the patients may be treated to reduce the mortality rate from EAC, or even prevent the onset of EAC. However, a targeted approach to finding at-risk patients is needed, so the at-risk patients may undergo such cancer screening. While some risk factors for developing EAC are known, such as gastroesophageal reflux disease (GERD), one or more of the risk factors may be unreported, or may be precursors to/indicative of other medical conditions unassociated with EAC. Because gastric cardia adenocarcinoma (GCA) shares many of the same risk factors as EAC, and is often clinically indistinguishable from EAC, the same issues that plague early detection of EAC or its precursors also apply to GCA.

Conventional computer-implemented tools and models to predict EAC and/or GCA generally require indications of potential risk factors such as GERD to be able to predict a risk of developing EAC and/or GCA. Unfortunately, EHRs may be missing data regarding one or more of the risk factors for a patient, causing the automated tools to be unable to generate any predictions or generate inaccurate predictions. For instance, the patient may not experience significant GERD symptoms or may take over-the-counter medication that controls the GERD symptoms, and thus the patient may not report their symptoms to their healthcare provider and/or may not be diagnosed with GERD, preventing a GERD diagnosis from appearing in the patient's EHRs. In other examples, the patient's GERD may be misdiagnosed due to the commonality of GERD symptoms with other medical issues, because the patient may not be asked about GERD symptoms by their healthcare provider, or because the healthcare provider may not deem the patient's GERD symptoms as important relative to the patient's other medical issues, any of which may cause an absence of a GERD diagnosis in the patient's EHRs. In addition, approximately one-half of patients who develop EAC and more of those with GCA deny having had prior significant GERD symptoms at all. Due to the lack of an indication of a risk factor like GERD in the patient's EHRs and the inability of conventional cancer prediction tools to make predictions in such instances despite the patient being at-risk for these types of cancers, the patient may never receive potentially life-saving treatment.

Therefore, there is an opportunity and need for improved systems and methods for predicting EAC and GCA.

### BRIEF SUMMARY

In one embodiment, the disclosure provides a computer-implemented method for predicting esophageal adenocarcinoma (EAC) and gastric cardia adenocarcinoma (GCA) using machine learning. The computer-implemented method may include obtaining, by one or more processors, an electronic health record (EHR) dataset including historical EHRs of historical patients; identifying, by the one or more processors, missing values of key predictors of EAC and/or GCA in at least a portion of the EHR dataset; generating, by the one or more processors, imputed values for at least a portion of the missing values of the key predictors using simple random sampling imputation; training, by the one or more processors, a model using a selected algorithm and a training dataset including at least a portion of the EHR dataset to generate a trained model, wherein: (i) the selected algorithm includes an extreme gradient boosting algorithm, (ii) the trained model includes multiple decision trees, and (iii) the training includes tuning (a) a maximum decision tree depth parameter, (b) a number of decision trees, (c) a percentage of the training dataset to train successive decision trees, and (d) a learning rate of the model, wherein the tuning achieves a greatest value of an area under a receiver operating characteristic curve associated with true positive rates and false positive rates of the model; obtaining, by the one or more processors, a patient EHR dataset of a patient; generating, by the one or more processors, a prediction associated with a risk of the patient developing EAC and/or GCA by applying the trained model to the patient EHR dataset; and providing, by the one or more processors, the prediction to a computing device to determine a patient treatment protocol. The method may include additional, less, or alternate functionality or actions, including those discussed elsewhere herein.

In a variation of the embodiment, the EHR dataset may indicate for the historical patients one or more of: sex, race, weight, body mass index (BMI), smoking status, Agent Orange exposure, International Classification of Diseases (ICD) codes, prescriptions, or laboratory results.

In another variation of the embodiment, the key predictors may include one or more of: smoking status, body mass index, or gastroesophageal reflux disease (GERD).

In yet another variation of the embodiment, the computer-implemented method may include applying, by the one or more processors, at least one label to at least a portion of the historical EHRs, the at least one label indicating one or more of: (i) a prescription for a proton pump inhibitor, (ii) a prescription for a histamine type 2 receptor antagonists, (iii) a complete blood count value, (iv) a comprehensive metabolic profile, (v) a cholesterol panel, (vi) a hemoglobin A1c value, (vii) a c-reactive protein value, or (viii) an ICD code associated one or more of: (a) heartburn, (b) reflux, or (c) a Charlson comorbidity score component not associated with cancer or metastatic cancer.

In still yet another variation of the embodiment, the computer-implemented method may include generating, by the one or more processors from the EHR dataset, prescription longitudinal summary data including one or more of: a maximum value, a largest increase value, or a total variation value of cumulative daily dosages and average daily dosages of proton pump inhibitors and histamine type-2 receptor antagonists in omeprazole and ranitidine equivalents, respectively, wherein the prescription longitudinal summary data is used to train the model.

In a variation of the embodiment, the computer-implemented method may include generating, by the one or more processors from the EHR dataset, laboratory longitudinal summary data including one or more of: a maximum value, a minimum value, a mean value, a largest increase value, a largest decrease value, or a total variation value of laboratory data included in the EHR dataset, wherein the laboratory longitudinal summary data is used to train the model.

In another variation of the embodiment, the EHR dataset may include one or more of: EHR data from at least 1 year before a cancer diagnosis of the historical patients, EHR data of the historical patients between ages of 18 and 90 at a time of the cancer diagnosis, or EHR data of the historical patients having at least 2 years of EHR data in a respective historical patient EHR dataset.

In yet another variation of the embodiment, one or more of: (i) the maximum decision tree depth parameter may indicate a maximum depth of 6 per decision tree; (ii) the number of the decision trees is 1,000; (iii) the percentage of the training dataset to train the successive decision trees may be 50%; or (iv) the learning rate of the model may be 0.05.

In still yet another variation of the embodiment, the computer-implemented method may include generating, by the one or more processors from the training dataset, a first dataset for training a plurality of models, a second dataset for selecting the model from the plurality of models, and a third dataset for testing the model.

In a variation of the embodiment, the prediction may include a metric associated with the risk; and based upon the metric exceeding a threshold, the patient treatment protocol includes a recommendation for a cancer screening.

In another variation of the embodiment, the metric may be a first metric and the cancer screening may include an upper endoscopy; or the metric may be a second metric and the cancer screening may include a non-endoscopic tissue sampling.

In another embodiment, the disclosure provides a system for predicting esophageal adenocarcinoma (EAC) and gastric cardia adenocarcinoma (GCA) using machine learning. The system may include one or more processors; and one or more non-transitory memories storing processor-executable instructions that, when executed by the one or more processors, cause the system to: obtain an electronic health record (EHR) dataset including historical EHRs of historical patients; identify missing values of key predictors of EAC and/or GCA in at least a portion of the EHR dataset; generate imputed values for at least a portion of the missing values of the key predictors using simple random sampling imputation; train a model using a selected algorithm and a training dataset including at least a portion of the EHR dataset to generate a trained model, wherein: (i) the selected algorithm includes an extreme gradient boosting algorithm, (ii) the trained model includes multiple decision trees, and (iii) to train the model includes tuning (a) a maximum decision tree depth parameter, (b) a number of decision trees, (c) a percentage of the training dataset to train successive decision trees, and (d) a learning rate of the model, wherein the tuning achieves a greatest value of an area under a receiver operating characteristic curve associated with true positive rates and false positive rates of the model; obtain a patient EHR dataset of a patient; generate a prediction associated with a risk of the patient developing EAC and/or GCA by applying the trained model to the patient EHR dataset; and provide the prediction to a computing device to determine a patient treatment protocol. The system may include additional, less, or alternate functionality, including that discussed elsewhere herein.

In yet another embodiment, a non-transitory computer readable medium having processor-executable instructions stored thereon that, when executed by one or more processors, cause the one or more processors to at least: obtain an electronic health record (EHR) dataset including historical EHRs of historical patients; identify missing values of key predictors of esophageal adenocarcinoma (EAC) and/or gastric cardia adenocarcinoma (GCA) in at least a portion of the EHR dataset; generate imputed values for at least a portion of the missing values of the key predictors using simple random sampling imputation; train a model using a selected algorithm and a training dataset including at least a portion of the EHR dataset to generate a trained model, wherein: (i) the selected algorithm includes an extreme gradient boosting algorithm, (ii) the trained model includes multiple decision trees, and (iii) to train the model includes tuning (a) a maximum decision tree depth parameter, (b) a number of decision trees, (c) a percentage of the training dataset to train successive decision trees, and (d) a learning rate of the model, wherein the tuning achieves a greatest value of an area under a receiver operating characteristic curve associated with true positive rates and false positive rates of the model; obtain a patient EHR dataset of a patient; generate a prediction associated with a risk of the patient developing EAC and/or GCA by applying the trained model to the patient EHR dataset; and provide the prediction to a computing device to determine a patient treatment protocol. The instructions may direct additional, less, or alternate functionality, including that discussed elsewhere herein.

Additional, alternate and/or fewer actions, steps, features and/or functionality may be included in an aspect and/or embodiments, including those described elsewhere herein.

### BRIEF DESCRIPTION OF THE DRAWINGS

The figures described below depict various aspects of the system and methods disclosed therein. It should be understood that each figure depicts one embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present aspects are not limited to the precise arrangements and instrumentalities shown, wherein:

FIG. 1 depicts a block diagram of an exemplary computing environment in which methods and systems for predicting esophageal adenocarcinoma and gastric cardia adenocarcinoma using machine learning are implemented, according to some embodiments.

FIG. 2 depicts an exemplary combined block and logic diagram for training a machine learning model, according to some embodiments.

FIG. 3A depicts an exemplary area under a receiver operating characteristic curve associated with discrimination of a prediction machine learning model for esophageal adenocarcinoma and gastric cardia adenocarcinoma, according to some embodiments.

FIG. 3B depicts an exemplary bar graph ranking the importance of variables associated with esophageal adenocarcinoma and gastric cardia adenocarcinoma in terms of the mean Shapley Additive Explanations, according to some embodiments.

FIG. 3C depicts an exemplary bar graph ranking the importance of variables associated with esophageal adenocarcinoma and gastric cardia adenocarcinoma in terms of the proportion of gain in information, according to some embodiments.

FIG. 3D depicts an exemplary display of a computing device including predictions of EAC and GCA for patients, according to some embodiments.

FIG. 4 depicts a flow diagram of an exemplary computer-implemented method for predicting esophageal adenocarcinoma and gastric cardia adenocarcinoma using machine learning, according to some embodiments.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

### Overview

Broadly speaking, the techniques of the present disclosure relate to predicting future EAC and GCA using machine learning (ML). The disclosed systems and methods may obtain an EHR dataset including historical EHRs of historical patients, identify missing values of key predictors of future EAC and/or GCA in at least a portion of the EHR dataset, and generate imputed values using simple random sampling imputation for at least a portion of the missing values of the key predictors. The disclosed techniques may include generating a trained machine leaning model including multiple decision trees. A training dataset including at least a portion of the EHR dataset, and an extreme gradient boosting algorithm may train the machine learning model. Training the model may include tuning a decision tree depth parameter associated with the length of the longest path from the root node to a leaf node of a decision tree of the machine learning model, a maximum number of decision trees of the machine learning model, tuning the percentage of the training dataset used to train successive decision trees, and/or tuning the learning rate of the machine learning model. Through tuning, the machine learning model may achieve a greatest value of an area under a receiver operating characteristic curve (AUROC) associated with a true positive rate and a false positive rate of the model. The systems and methods may obtain a patient's EHR dataset, generate a prediction of the patient developing EAC and/or GCA by applying the trained machine learning model to the patient EHR dataset, and provide the prediction to a computing device to determine a patient treatment protocol.

The disclosed techniques improve the technical field of predicting EAC and GCA. Conventional EAC and GCA prediction models require data values, such as key predictors, be present in the EHR dataset to provide accurate predictions, or any predictions. By imputing the missing values, this problem is alleviated, allowing for the prediction of EAC and GCA when EHR data is missing values required for the prediction, thereby improving the technical field of predicting EAC and GCA. In accordance with the above, and with the disclosure herein, the present disclosure includes improvements in computer functionality and/or improvements to other technologies at least because the techniques provide, e.g., identifying missing values of key predictors of EAC and/or GCA in at least a portion of the EHR dataset, generating imputed values for at least a portion of the missing values of the key predictors using simple random sampling imputation, and training a model using an extreme gradient boosting algorithm and a training dataset including at least a portion of the EHR dataset to generate a trained model including multiple decision trees.

As used herein, the terms “predict,” “prediction,” and the like as used herein refer to a prediction of future risk of developing EAC and/or GCA, rather than diagnosing existing EAC and/or GCA. Similarly key ““predictors” and similar terms as used herein refer to key predictors of future EAC and/or GCA rather than existing EAC and/or GCA.

### Exemplary Computing Environment

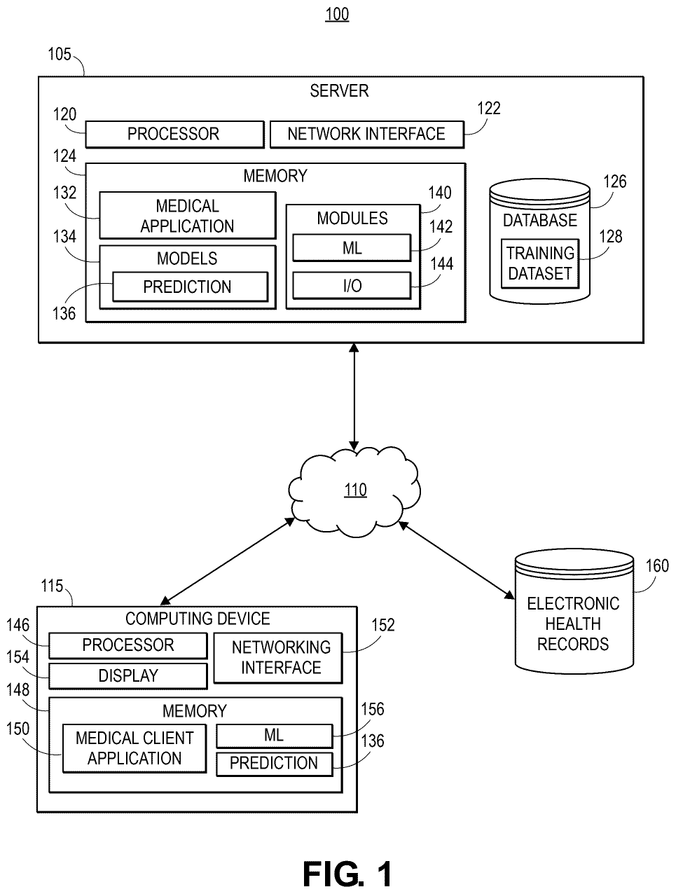

FIG. 1 depicts an exemplary computing environment 100 associated with predicting EAC and GCA using machine learning. The computing environment 100 may include at least one server 105 and at least one computing device 115 communicatively coupled via a network 110. Although FIG. 1 depicts certain entities, components, equipment, and/or devices, it should be appreciated that additional, fewer, and/or alternate entities, components, equipment, and/or devices are envisioned.

The at least one server 105 may perform the at least some of the disclosed functionalities and techniques associated with predicting EAC and GCA using machine learning. The server 105, referred to at times more generically as a “computing device” or “device,” may be part of a cloud network or may otherwise communicate with other hardware or software components within one or more cloud computing environments to send, retrieve, or otherwise analyze data or information described herein. In some embodiments, the computing environment 100 may comprise an on-premises computing environment, a multi-cloud computing environment, a public cloud computing environment, a private cloud computing environment, and/or a hybrid cloud computing environment. In one example, the server 105 may host one or more services (e.g., patient cancer predictions) in a public cloud computing environment (e.g., Amazon Web Services (AWS), Google Cloud, IBM Cloud, Microsoft Azure, etc.). The public cloud computing environment may be a traditional off-premises cloud (i.e., not physically hosted at a location owned/controlled by an entity offering services provided via the server 105). Alternatively, or in addition, aspects of the public cloud may be hosted on-premises at a location owned/controlled by the entity. The public cloud may be partitioned using visualization and multi-tenancy techniques and/or may include one or more of software-as-a-service (SaaS), infrastructure-as-a-service (IaaS) and/or platform-as-a-service (PaaS). In one aspect, the server 105 may include a client-server platform technology such as ASP.NET, Java J2EE, Ruby on Rails, Node.js, a web service or online API, responsive for receiving and responding to electronic requests.

The server 105 may include a network interface 122. The network interface 122 may allow the server 105 to communicate over the network 110 via any suitable wired and/or wireless connection, e.g., using any suitable network interface controller(s) of the network interface 122. The network interface 122 may include one or more transceivers (e.g., WWAN, WLAN, and/or WPAN transceivers) functioning in accordance with IEEE reference standards, 3GPP reference standards, and/or other reference standards that may be used in receipt and transmission of data via external/network ports of the server 105 connected to computer network 110.

The server 105 may include at least one processor 120. The processor 120 may include one or more suitable processors (e.g., central processing units (CPUs) and/or graphics processing units (GPUs)). The processor 120 may be communicatively coupled to a memory 124 via a computer bus (not depicted) that transmits electronic data, data packets, or otherwise electronic signals to and from the processor 120 and the memory 124 in order to execute, implement or perform the machine-readable instructions, methods, processes, elements, or limitations, as illustrated, depicted, or described for the various flowcharts, illustrations, diagrams, figures, and/or other disclosure herein. The processor 120 may interface with the memory 124 to execute an operating system, computing instructions contained therein, and/or to access other services/aspects. For example, the processor 120 may interface with the memory 124 via the computer bus to create, read, update, delete, or otherwise access or interact with the data stored in the memory 124, database 126, and/or another source of data.

The memory 124 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 124 may store the operating system (e.g., Microsoft Windows, Linux, UNIX, etc.) capable of facilitating the functionalities, apps, methods, or other software as described herein. The memory 124 may store one or more sets of non-transitory, computer-executable instructions that, when executed, cause the server 105 to perform certain functions.

In general, a computer program or computer-based product, application, or code (e.g., ML models, or other computing instructions described herein) may be stored on a computer usable storage medium, or tangible, non-transitory computer-readable medium (e.g., reference random access memory (RAM), an optical disc, a universal serial bus (USB) drive, or the like) having such computer-readable program code or computer instructions embodied therein. The computer-readable program code or computer instructions may be installed on, or otherwise adapted to be, executed by the processor 120 (e.g., working in connection with the respective operating system in the memory 124) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective C, Java, Scala, ActionScript, JavaScript, HTML, CSS, XML, etc.).

The server 105 may include, and/or be communicatively coupled to (e.g., via the network 110), at least one electronic database 126. The database 126 may include a relational database, such as Oracle, DB2, MySQL, a NoSQL database such as MongoDB, and/or another other suitable database. The database 126 may store data and/or datasets as discussed herein, such as ML model training dataset 128, ML models, ML model input and/or output data, EHR datasets of one or more patients, and/or any other suitable data. A dataset may include one or more types of data, records, files, etc. The terms “data” and “dataset” may be used interchangeably herein.

The memory may store a medical application 132 that, when executed by the processor 120, performs one or more functions associated with predicting EAC and/or GCA, such as obtaining the EHR dataset of a patient, applying a prediction model 136 to the patient's EHR dataset, providing the prediction to the computing device 115, determining a patient treatment protocol (e.g., recommending and/or scheduling a cancer screening, a future check-up, laboratory work, etc.). In some embodiments, a user executes the medical application 132, such as a local user of the server 105 (e.g., via a user interface of the server 105), a user remotely accessing the medical application 132 over the network 110 (e.g., via a medical client application 150 of the computing device 115), and/or any other suitable user. In some embodiments, the medical application 132 may be configured to execute automatically (e.g., according to a schedule, continuously, in response to a trigger event such as receiving an EHR dataset, etc.).

The memory 124 or other suitable storage (e.g., the database 126) of the computing environment 100 may store one or more ML models 134, routines, algorithms, or other elements (collectively “models” or “ML models”). The ML models 134 may be, or include, computer-executable instructions that when executed (e.g., by the processor 120 of the server 105, by the computing device 115) cause the one or more of the ML models 134 to receive one or more inputs, and generate and/or store (e.g., in the memory 124, the database 126) one or more outputs. Further, the processor 120 should be understood to retrieve/access from the memory 124 and/or the database 126 any data necessary to perform the executed instructions (e.g., data required as an input to one of the ML models 134), and to store in the memory 124 and/or the database 126 the intermediate results and/or output of any executed instructions.

The ML models 134 may include a prediction ML model 136, also referred to at times as a “prediction model.” In at least some embodiments, the prediction model 136 may be, or include, an ensemble of multiple decision trees, such as one thousand decision trees. In at least some embodiments, the prediction ML model 136 may be trained using extreme gradient boosting (XGBoost). An ML module 142 may train the prediction model 136 to receive an EHR dataset of a patient as an input and generate a prediction associated with a risk of the patient developing EAC and/or GCA as an output. In at least some embodiments, the prediction may include one or more metrics associated with the risk of developing EAC and/or GCA, and/or other suitable information associated with predicting EAC and/or GCA. The metric may be and/or include a score, a ranking, a percentage, a rating, and/or any other suitable metric. In one example, the metric may be a score predicting the risk of EAC and/or GCA per 100,000 individuals, such as 900 EAC/GCA cancers per 100,000 individuals. In another example, the score may be extrapolated over a time frame, such as a predicted one year incidence of EAC/GCA of about 64 per 100,000 individuals per year. In yet another example, the score may be converted to a percentage of an absolute risk, such as 0.02%. The prediction may be used, for example, to determine a patient treatment protocol (e.g., by the patient's healthcare provider).

The training dataset 128 may include historical information associated with training the prediction model 136. The training dataset 128 may include historical EHR datasets for a plurality of historical patients. The historical EHR datasets may include and/or otherwise provide an indication of diagnoses of EAC and/or GCA for the plurality of historical patients. During training, associations and relationships may be made between information in the historical EHR datasets (e.g., historical patient demographics, laboratory values, ICD codes, smoking status, Agent Orange exposure, BMI, prescriptions, etc.) and diagnoses of EAC and/or GCA for the historical patients, for example to identify key predictors of EAC and/or GCA. The training dataset 128 may include historical predictions of EAC and/or GCA, such as historical predictions generated by the prediction model 136 or other suitable predictions of EAC and/or GCA. The historical EHR datasets may indicate (e.g., based on historical EHRs provided after the historical predictions) whether the historical predictions were accurate, for example whether a historical patient predicted as at-risk for developing GCA did develop GCA after the prediction was made. The historical EHR datasets and historical predictions of the training dataset 128 may be used, for example, to retrain the prediction model 136 to provide more accurate results based upon the retraining. In at least some embodiments, the training dataset 128 may include a first dataset for training a plurality of models, a second dataset for selecting the model from the plurality of models, and a third dataset for testing the model.

The memory 124 may store one or more computing modules 140, implemented as respective sets of computer-executable instructions (e.g., one or more source code libraries), as described herein. Although FIG. 1 depicts the ML models 134 as part of the memory 124, one or more of the ML models 134 may be considered as a computing module 140, may be stored in the database 126, may be stored on a device accessible via the network 110, etc.

The computing modules 140 may include the ML module 142. In some embodiments, ML models (e.g., the ML models 134) may be applied by the ML module 142, which may include, but are not limited to linear or logistic regression algorithms, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of ML, such as supervised learning, unsupervised learning, and reinforcement learning. In one aspect, the ML based algorithms may be included as a library or package executed on server(s) 105. For example, libraries may include the TensorFlow based library, the Pytorch library, and/or the scikit learn Python library.

In one embodiment, the ML module 142 employs supervised learning, which involves identifying patterns in existing data to make predictions about subsequently received data. Specifically, the ML module 142 is “trained” using training dataset (e.g., the training dataset 128), which includes exemplary inputs and associated exemplary outputs. Based upon the training dataset, the ML module 142 may generate a predictive function which maps outputs to inputs and may utilize the predictive function to generate ML outputs based upon data inputs. The exemplary inputs and exemplary outputs of the training dataset may include any of the data inputs or ML outputs described herein. In the exemplary embodiments, a processing element may be trained by providing it with a large sample of data with known characteristics or features.

In another embodiment, the ML module 142 may employ unsupervised learning, which involves finding meaningful relationships in unorganized data. Unlike supervised learning, unsupervised learning does not involve user-initiated training based upon exemplary inputs with associated outputs. Rather, in unsupervised learning, the ML module 142 may organize unlabeled data according to a relationship determined by at least one ML method/algorithm employed by the ML module 142.

In yet another embodiment, the ML module 142 may employ reinforcement learning, which involves optimizing outputs based upon feedback from a reward signal. Specifically, the ML module 142 may receive a user-defined reward signal definition, receive a data input, utilize a decision making model to generate the ML output based upon the data input, receive a reward signal based upon the reward signal definition and the ML output, and alter the decision making model so as to receive a stronger reward signal for subsequently generated ML outputs. Other types of ML may also be employed, including deep or combined learning techniques.

The ML module 142 may receive labeled data at an input layer of a model having a networked layer architecture (e.g., an artificial neural network, a convolutional neural network, etc.) for training one or more ML models. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models.

The ML module 142 may comprise a set of computer-executable instructions to implement functionality such as loading, configurating, initializing, operating, and/or storing (e.g., in the memory 124, the database 126) the ML models 134. Once trained, one or more of the trained ML models 134 may be operated in inference mode, whereupon when provided with de novo input that the model has not previously been provided, the model may output one or more predictions, classifications, etc., as described herein.

In operation, the ML module 142 may access the memory 124, the database 126, and/or any other data source for the training dataset (e.g., training dataset 128) suitable to generate one or more ML models, such as the prediction model 136. The training dataset may be sample data with assigned relevant and comprehensive labels (classes or tags) used to fit the parameters (weights) of the ML model with the goal of training it by example. In one aspect, once an appropriate ML model is trained and validated to provide accurate predictions and/or responses, the trained ML model may be loaded into the ML module 142 at runtime to process input data and generate output data.

While various embodiments, examples, and/or aspects disclosed herein may include training and generating the ML models 134 for the server 105 to load at runtime, one or more appropriately trained ML models may already exist (e.g., stored in the memory 124, the database 126) such that the server 105 may load the existing trained ML model 134 at runtime. The server 105 may retrain, fine-tune, update and/or otherwise alter an existing ML model 134 before and/or after loading the ML model 134 at runtime. Although the ML model 134 may be described as being trained and operated (e.g., via ML module 142) on the server 105, in at least one embodiment the ML model 134 may be trained on the server 105 (e.g., or other computing device), and operated on another server (or another computing device).

In one aspect, the computing modules 140 may include an input/output (I/O) module 144, comprising a set of computer executable instructions implementing communication functions. The I/O module 144 may include a communication component configured to communicate (e.g., send and receive) data via one or more external/network port(s) to one or more networks or local terminals, such as the network 110 described herein. The I/O module 144 may include or implement a user interface configured to present information to an administrator, operator or other user, and/or receive inputs from the user, such as via a touchscreen display. The I/O module 144 may facilitate I/O components (e.g., ports, capacitive or resistive touch sensitive input panels, keys, buttons, lights, LEDs), which may be directly accessible via, or attached to, the server 105 and/or may be indirectly accessible via, or attached to, another device. According to one aspect, a user may access the server 105 via a user interface to input and/or review data/information, initiate ML model training via the ML module 142, and/or perform other functions.

The network 110 may include one or more networks, including a local area network (LAN), wide area network (WAN), the Internet, a combination thereof, and/or any other suitable network. Generally, the network 110 enables bidirectional communication between the server 105, the computing device 115, and other components and/or devices of the computing environment 100. In some embodiments, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, or the like. Additionally, or alternatively, the network 110 may comprise one or more routers, wireless switches, or other such wireless connection points communicating to the components of the computing environment 100 via wireless communications based upon any one or more of various wireless standards, including by non-limiting example, IEEE 802.11 a/ac/ax/b/c/g/n (Wi-Fi), Bluetooth, and/or the like.

The computing environment 100 may include at least one computing device 115, also referred to as a “user device.” The computing device 115 may include a desktop computer, laptop computer, terminal, server, a mobile device, a wearable, augmented reality glasses/headsets, virtual reality glasses/headsets, mixed or extended reality glasses/headsets, and/or other suitable computing device. The computing device 115 may include a processor 146 (e.g., the processor 120) and a memory 148 (e.g., the memory 124) for storing and executing one or more applications, modules, computer-executable instructions, etc. The computing device 115 may further include a network interface 152 (e.g., the network interface 122) and a display 154 (e.g., LCD, LED, OLED, head-mounted, etc.). The computing device 115 may access services, devices, and/or components of the computing environment 100 via the network 110. In some embodiments, the computing device 115 transmits and/or receives information/data with the server 105 and/or other components of the computing environment 100. For example, the computing device 115 may receive from the server 105 via the network 110 the prediction associated with a risk of the patient developing EAC and/or GCA. In at least some embodiments, the computing device 115 may be associated with the patient and/or a healthcare provider (e.g., doctor) of the patient.

The memory 148 of the computing device 115 may store the medical client application 150. The medical client application 150 may be configured to provide the same and/or similar functionality as the medical application 132, and/or be communicatively connected (e.g., via the network 110) to the medical application 132 to provide the functionality of the medical application 132 to the user of the medical client application 150. In one example, the medical client application 150 may be a mobile device application used by the patient and/or a healthcare provider of the patient to predict the patient's risk of EAC and/or GCA. In another example, the medical client application 150 may communicate with the medical application 132 via the network 110 to generate the risk of EAC and/or GCA remotely at the server 105.

The memory 148 of the computing device 115 may store an ML module 156 (e.g., the ML module 142) that may execute the prediction model 136 stored in the memory 148. In one example, the computing device 115 may be a server associated with a healthcare provider. The server 105 may train the prediction model 136, the computing device 115 may retrieve the trained prediction model 136 from the server 105 via the network 110, and store the prediction model 136 locally in the memory 148. The healthcare provider may use the prediction model 136 to make predictions of EAC and/or GCA for the healthcare provider's patients. The healthcare provider may execute the medical client application 150. The medical client application 150 may be configured to obtain EHR datasets from an EHR database 160 via the network 110, cause the ML module 156 to load the prediction model 136, provide the EHR dataset of a patient to the prediction model 136, and receive the prediction of EAC/GC for the patient from the prediction model 136 as a result.

The computing environment 100 may include the EHR database 160 communicatively coupled (e.g., via the network 110) to one or more components and/or devices (e.g., the server 105, the computing device 115) of the computing environment 100. In some embodiments, the EHR database 160 may store EHR datasets of one or more patients, such as patients of one or more healthcare providers. It should be understood that although the systems, methods and techniques disclosed herein generally describe predictions of the risk of EAC and/or GCA for a single patient, the systems, methods and techniques may be applied to make predictions of EAC and/or GCA for a plurality of patients (e.g., hundreds, thousands, etc.).

It should also be understood that, while the computing environment 100 is shown in FIG. 1 to include one each of the server 105, the network 110, the computing device 115, and the EHR database 160, different numbers of servers 105, networks 110 computing devices 115, and/or EHR database 160 may be utilized. In one example, the computing environment 100 may include hundreds of computing devices 115 and EHR databases 160 associated with different healthcare providers, all of which may be interconnected via the network 110 to provide predictions for the patients of the various healthcare providers.

The computing environment 100 may include additional, fewer, and/or alternate components, and may be configured to perform additional, fewer, or alternate actions, including components/actions described herein. For example, although the server 105 is shown in FIG. 1 as including one instance of the processor 120, the memory 124 and the database 126, various aspects may include the server 105 implementing any suitable number of any of the components shown in FIG. 1 and/or omitting any suitable ones of the components shown in FIG. 1. In another example, at least some of the data described as being stored in the EHR database 160 may be stored in the database 126, and therefore the EHR database 160 may be omitted. Furthermore, it should be appreciated that additional and/or alternative connections between components shown in FIG. 1 may be implemented. As just one example, server 105 may be connected to the database 126 via the network 110 rather than being locally connected to one another via a direct connection as illustrated in FIG. 1.

### Exemplary Ml Model Training

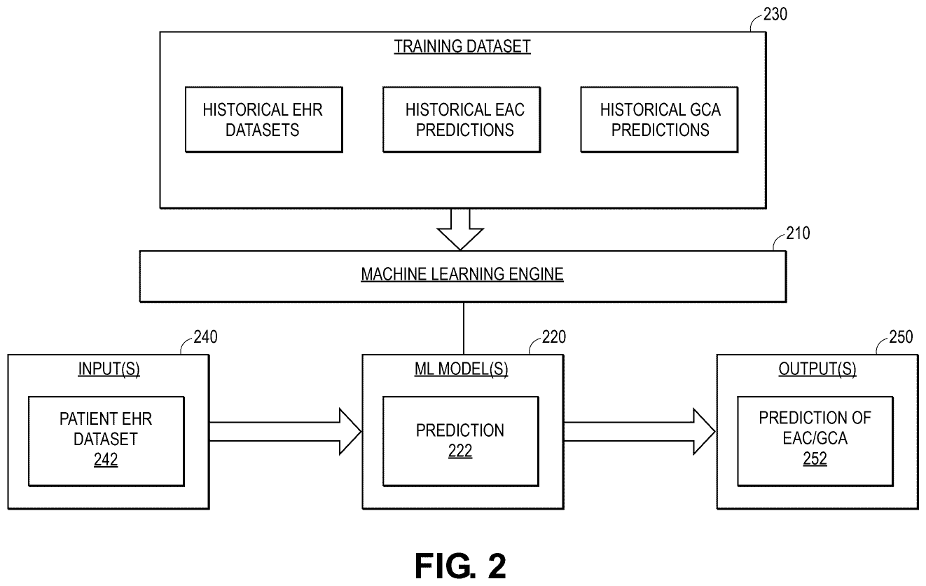

FIG. 2 depicts a combined block and logic diagram for training a machine learning model, according to some embodiments. More specifically, an ML engine 210 (e.g., the ML module 142) trains an ML model 220 (e.g., the prediction model 136) using a training dataset 230 (e.g., the training dataset 128). The trained ML models 220 are applied to, and/or receive, at least one input 240 and generate at least one output 250.

The ML engine 210 may include one or more hardware and/or software components to obtain, create, (re) train, operate, fine-tune, and/or store the ML model 220. A computing device (e.g., the server 105, the computing device 115), may obtain and/or have available (e.g., stored in the database 126, 160) the training datasets 230, at least a portion of which may be used for model creation, training, retraining and/or fine-tuning (generally referred to herein as “training”). In at least one aspect, at least some of the training dataset 230 may be labeled to aid in training the ML model 220. The ML engine 210 may process and/or analyze the training dataset 230 to learn associations and/or relationships in the training dataset 230, and configure the ML model 220 to process the training dataset 230 such that when the ML model 220 receives one or more inputs 240, the ML model 220 generates appropriate output(s) 250. The ML model 220 may be trained via regression, k-nearest neighbor, support vector machines, random forest algorithms, although any type of applicable ML algorithm and/or training may be used, including training using one or more of supervised learning, unsupervised learning, semi-supervised learning, and/or reinforcement learning.

In at least one aspect, the ML model 220 may be considered as successfully trained when able to achieve one or more metrics (e.g., a score indicating accuracy) with satisfying values associated with its performance when processing the training dataset 230. Once trained, the ML engine 210 may load the ML model 220 at runtime to perform operations on one or more data inputs 240 to produce one or more desired data outputs 250.

In at least some embodiments, the ML model 220 may be, and/or include, the prediction ML model 222 (e.g., the prediction model 136) that is trained to generate a prediction 252 associated with a risk of the patient developing EAC and/or GCA as an output 250, based on receiving (e.g. from the EHR database 160) a patient EHR dataset 242 as an input 240. The training dataset 230 for the prediction ML model 222 may include historical EHR datasets of historical patients, historical predictions of EAC for the historical patients, historical predictions of GCA for the historical patients, and/or any other suitable training data. The (historical) patient EHR datasets may include and/or indicate for the associated patient their sex, race, weight, demographics, BMI, smoking status/history, and/or Agent Orange exposure. The EHR dataset may include and/or indicate diagnoses, and/or indications of health conditions of the patients (e.g., heartburn, GERD, EAC, GCA, esophageal conditions, chronic obstructive pulmonary disease (COPD), etc.), prescription information (e.g., quantity, dosage, refills of prescriptions such as proton pump inhibitors, histamine type 2 receptor antagonists, omeprazole, ranitidine, etc.), laboratory results (e.g., complete blood count, comprehensive metabolic profile, cholesterol panels, hemoglobin Alc, and C-reactive protein), diagnoses (e.g.,), medical procedures (e.g., upper endoscopy, non-endoscopic tissue sampling), and/or any other suitable EHR information and/or data.

In at least some embodiments, the types of information contained in the EHRs for one patient may not be included in the EHRs of another patient. For example, a first patient's EHR record may include their BMI, while a second patient's EHR may not include their BMI for example when the second patient's BMI was not determined by their healthcare provider. In instances of missing values in an EHR, the missing values may be imputed using one or more methodologies, such as median sampling, simple random sampling, multiple random sampling, multiple imputation using chained equations, and/or other imputation methods. In at least some embodiments, the missing values in the EHR dataset may be imputed using simple random sampling imputation.

In at least some embodiments, at least a portion of the EHR training dataset may be labeled with one or more labels, as described above. For example, the labeling may indicate one or more of: (i) a prescription for a proton pump inhibitor, (ii) a prescription for a histamine type 2 receptor antagonists, (iii) a complete blood count value, (iv) a comprehensive metabolic profile, (v) cholesterol panel values, (vi) a hemoglobin A1c value, (vii) a c-reactive protein value, (viii) an ICD code associated one or more of: (a) heartburn, (b) reflux, or (c) a Charlson comorbidity score component not associated with cancer or metastatic cancer (e.g., ICD codes for heart failure, kidney disease, COPD, etc.), and/or any other suitable labels.

In at least some embodiments, dosages of prescriptions, such as daily dosages of omeprazole, ranitidine, and equivalents, may be converted to prescription longitudinal summaries, including maximum, largest increase, total variation, and/or other suitable metrics. In at least some embodiments, laboratory results, such as the complete blood count, comprehensive metabolic profile, cholesterol panels, hemoglobin Alc, and C-reactive protein may be converted to laboratory longitudinal summaries, including maximum, minimum, and mean values, largest increase, largest decrease, total variation and/or other suitable metrics. The prescription and/or laboratory longitudinal summaries may be used to label the training data 230, used as the training data 230, used during training to identify/associate key predictors with the development of EAC and/or GCA, etc.

The ML engine 210 may train the prediction model 222 to learn associations and/or relationships between information in historical EHR datasets and the risk of EAC and/or GCA such that when receiving the EHR dataset 242 of a patient as the input 240, the prediction model 222 can successfully generate the prediction 252 of the risk of EAC and/or GCA for the patient as the desired output 250. In at least some embodiments, training the prediction ML model 222 may include using an extreme gradient boosting algorithm. In at least some embodiments, the prediction ML model 222 may be, or include, an ensemble of decision trees.

Training of the prediction ML model 222 using the EHR datasets of historical patients may indicate associations between, for example, diagnoses of EAC and/or GCA and historical patient characteristics such as age, race, sex, instances of GERD and/or COPD, and/or laboratory result values associated with white blood cell count, sodium and hematocrit. The historical patient characteristics may be key indicators of developing EAC and/or GCA, such that when the trained prediction ML model 222 receives EHR datasets of new patients that include the key indicators, the prediction ML model 222 can generate accurate predictions of the risk of the new patient developing EAC and/or GCA.

The prediction ML model 222 may be considered as successfully trained when able to achieve one or more metrics, such as an area under a receiver operating characteristic curve with satisfying values. The one or more metrics, or successful model training, may be achieved through tuning the prediction ML model 222. In one example, the maximum number of decision trees comprising the prediction ML model 222 may be modified at one or more times during tuning of the prediction ML model 222. In another example, the percentage of the training dataset 230 used to train successive decision trees may be modified at one or more times during tuning of the prediction ML model 222. In yet another example, the learning rate of the prediction ML model 222 may be adjusted one or more times during tuning of the prediction ML model 222. In at least some embodiments, the prediction ML model 222 with 1,000 decision trees each having a maximum decision tree depth of 6 per tree, using 50% of the training dataset 230 to train the successive decision trees, and with the learning rate of 0.05 may achieve a greatest value of an AUROC, the axes of the AUROC including a true positive rate, also referred to as sensitivity (e.g., on the y-axis) and a false positive rate, also referred to as 1-specificity (e.g., on the x-axis) of the prediction ML model 222.

Select information from the historical EHR datasets may be used during training. In at least some embodiments, select information of the EHR training dataset used for training. In one example, the EHR training dataset may be limited to EHR data from at least 1 year before a cancer diagnosis of the historical patient, random index date for patients without cancer, and/or any other suitable data limitation. In another example, the EHR training dataset may be limited to EHR data of historical patients between the ages of 18 and 90 at the time of the cancer diagnosis. In yet another example, the EHR training dataset may be limited to EHR data of the historical patients having at least 2 years of EHR data in a respective historical patient EHR dataset, although any other suitable subset of the EHR dataset may be used as training data. Selecting subsets of the historical EHR data to use during training may provide for improved training and/or improved output of the prediction ML model 222.

In at least some embodiments, Training the prediction ML model 222 may include using different subsets of the training dataset 230. In at least some embodiments, training the prediction ML model 222 may include a model training phase using a first portion of the training dataset 230, a model selection phase using a second portion of the training dataset 230, and a model testing phase using a third portion of the training dataset 230.

The server and/or the ML engine 210 may update the training dataset 230 at one or more times. The ML model 220 may be retrained using the updated training dataset 230, the retrained/updated ML model 220 may be stored in memory, and subsequently executed to generate more accurate outputs 250 based upon the retraining. The retraining process may cause the output 250 of the ML model 220 to improve over time. For example, based upon receiving a patient's EHR dataset 242, the prediction ML model 222 generates the prediction 252 for a patient that indicates a risk of developing EAC and/or GCA and also includes a recommendation of an upper endoscopy cancer screening (e.g., a patient treatment protocol), although any other suitable treatment and/or screening modality may be recommended. The server and/or ML engine 210 may store (in the memory 124 or the database 126) the patient's EHR dataset 242 and the prediction 252 as updated training data. The server may also obtain and store as updated training data new EHRs of the patient created after the prediction is made (e.g., EHRs of the patient over the next year), as the new EHRs may indicate if the patient received the upper endoscopy cancer screening, whether the upper endoscopy cancer screening indicated an occurrence of EAC and/or GCA in the patient, and/or whether the patient is eventually diagnosed with EAC and/or GCA. The updated EHRs may indicate whether the prediction 252 was accurate, whether the upper endoscopy cancer screening or some other patient treatment protocol should be recommended in similar circumstances in the future, etc. The prediction ML model 222 may be updated during retraining using the updated training data, so that the prediction ML model 222 may improve its predictions 252.

Although the prediction ML model 222 may be described as being trained and operated on the same computing device (e.g., a server), it should be understood that a first computing device may train the prediction ML model 222, and a second computing device may operate the prediction ML model 222.

It should be expressly understood that the disclosed techniques and system are not for generic application of known machine learning methods to new datasets, nor do they merely automate existing prediction practices. In contrast with conventional approaches that may simply apply an out-of-the-box machine learning framework-such as an unmodified gradient boosting model—to clinical datasets without specific adaptation, techniques disclosed herein involve a purposeful and multifactorial fine-tuning of the predictive model. Specifically, in some embodiments, the disclosed techniques include concurrent and iterative tuning of key model parameters, including (a) a maximum decision tree depth parameter, (b) a number of decision trees, (c) a percentage of the training dataset to train successive decision trees, and/or (d) a learning rate of the model, with such tuning directed towards achieving a particularly high area under a receiver operating characteristic (AUROC) curve relevant to the clinical discriminant task of predicting EAC and GCA risk. This contrasts sharply with generic practices that may rely on default settings or the adjustment of a single hyperparameter in isolation. By systematically optimizing these multiple parameters in the context of the extreme gradient boosting algorithm, techniques disclosed herein result in a model configuration tailored to the unique statistical and clinical characteristics of the imputed, feature-rich EHR dataset, producing a trained model with technical capabilities that are not available from conventional, merely “plug-and-play” machine learning models.

Further, as disclosed herein, the training methodology is neither routine nor a mere data substitution problem. The multivariable, synergetic tuning process takes into account interactions and dependencies between several model parameters—for example, recognizing that the optimal depth of each decision tree may vary as the number of decision trees or the learning rate is modified, and vice versa. During training, in some embodiments, the maximum decision tree depth parameter, the total number of decision trees, the fraction of the training dataset allocated to each successive tree, and/or the learning rate are not simply optimized individually in a vacuum; rather, they may be jointly tuned (for example, through grid search or other multi-parameter optimization procedures), directly in view of the AUROC measured on clinically-labeled, imputed datasets. This process creates a trained model with a particular structure and set of weights that are functionally and statistically optimized for detecting EAC and GCA risk patterns in real-world, incomplete EHR data. Such a configuration may not be achieved by merely applying available ML techniques to the present data—it emerges only from the disclosed harmonized tuning process, which dynamically exploits complex data interrelationships and validation feedback specific to the medical prediction objective.

As a result, the trained model as disclosed herein possesses a technical configuration and operational profile that are neither generic nor conventional. In some embodiments, the structural and algorithmic characteristics-such as the selected combination of parameter values (e.g., a maximum depth per decision tree, number of trees, percent data sampling per tree, and a learning rate)—are not arbitrary; rather, they are empirically determined and justified by their role in maximizing discriminatory accuracy as quantified by AUROC for the medically meaningful outcome of EAC/GCA prediction. The resulting model thus reflects a non-obvious, non-generic, and computer-implemented innovation that is particularly well-adapted to solving the technical problems posed by missing data, heterogeneous predictors, and the clinical need for actionable risk stratification in population-scale EHRs. Techniques disclosed herein, therefore, represent a specific, non-routine application and adaptation of machine learning technology that achieves technical improvements in cancer risk prediction unavailable through conventional or generic ML deployments.

### Exemplary Prediction of Esophageal Adenocarcinoma and Gastric Cardia Adenocarcinoma Using Machine Learning

In one embodiment, a Model Provider trains a prediction ML model (e.g., the prediction model 136, the prediction ML model 222) to provide predictions of EAC and/or GCA. The Model Provider obtains a plurality of EHRs from one or more databases (e.g., the EHR database 160) to use as training data (e.g., the training dataset 128, the training dataset 230) for the prediction ML model. For example, one source of training data may be EHRs of veterans provided by the Veterans Health Administration. The Model Provider may identify missing values in one or more of the EHRs comprising the EHR dataset, such as values of known key predictors (e.g., smoking status, body mass index, or GERD) of EAC and/or GCA in at least a portion of the EHR dataset. The Model Provider may impute values for at least some of the missing values using simple random sampling imputation, although other suitable imputation methods may be used.

In at least some embodiments, the Model Provider may further process the EHR dataset before training the model. In one example, the Model Provider labels at least some of the data of the EHR dataset. The EHR dataset (e.g., individual EHRs) may include labels indicating one or more of: (i) a prescription for a proton pump inhibitor, (ii) a prescription for a histamine type 2 receptor antagonists, (iii) a complete blood count value, (iv) a comprehensive metabolic profile, (v) a cholesterol panel, (vi) a hemoglobin A1c value, (vii) a c-reactive protein value, or (viii) an ICD code associated one or more of: (a) heartburn, (b) reflux, or (c) a Charlson comorbidity score component not associated with cancer or metastatic cancer, although any other suitable label may be used. In another example, the EHR dataset may be used to generate summary data, such as prescription longitudinal summary data and/or prescription longitudinal summary data as previously described. In yet another example, the EHR dataset may be processed to only include data meeting certain criteria suitable for trainings, such as data-limited data from at least 2 years before a cancer diagnosis of the associated patient; age-limited data such as EHR data of patients between ages of 18 and 90 at a time of a cancer diagnosis; data limited based upon the quantity of data such as EHR data of patients having at least 2 years of EHR data; and/or any other suitable criteria.

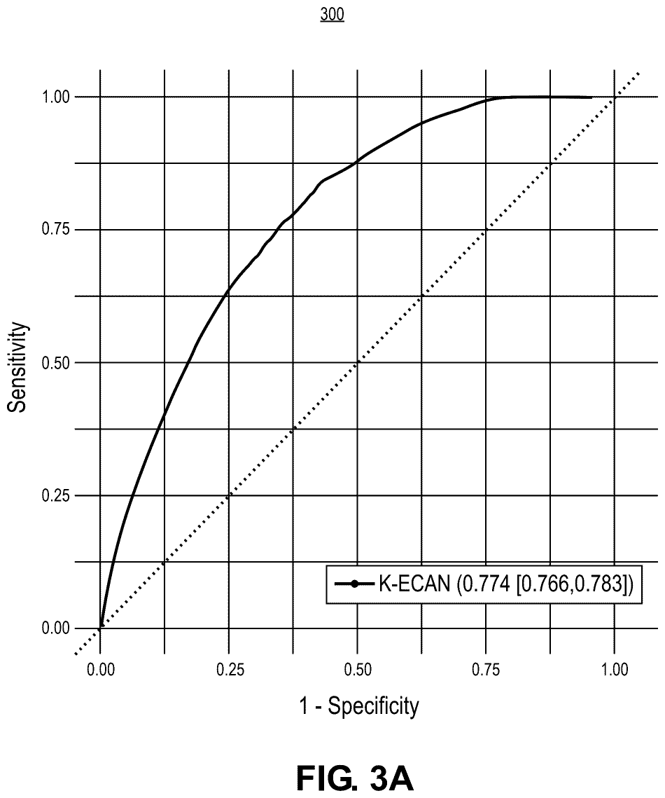

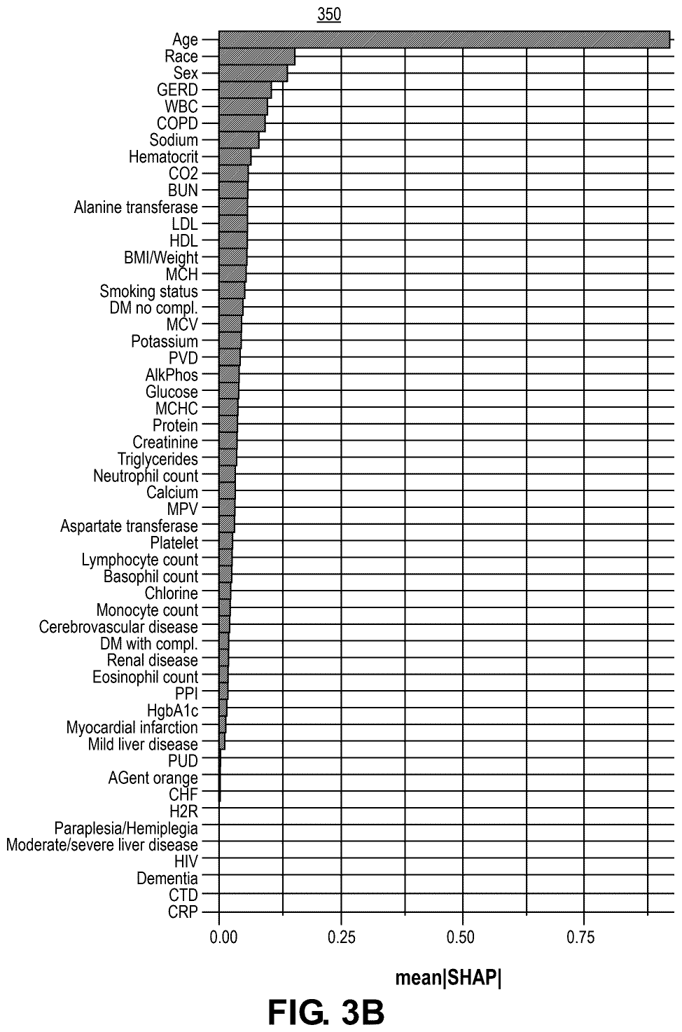

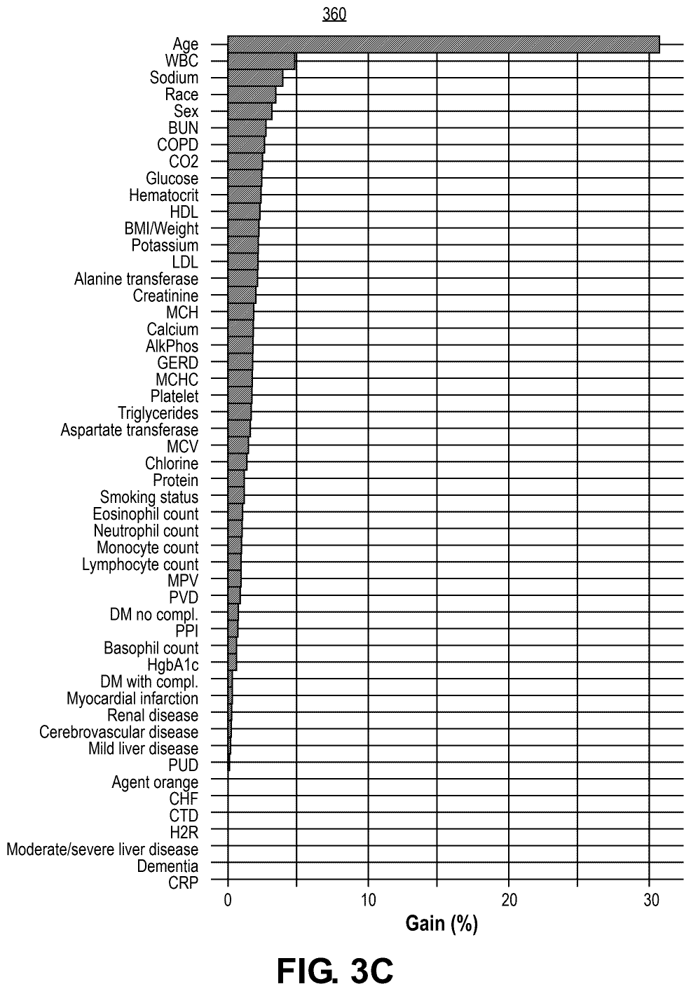

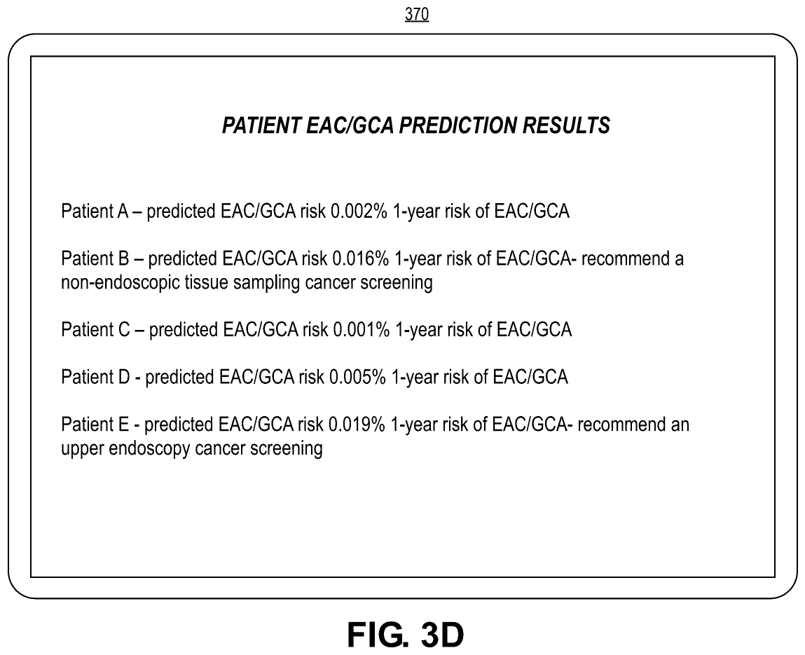

The Model Provider may use a server (e.g., the server 105) to train the prediction ML model (e.g., via the ML module 142, the ML engine ML engine 210) using an extreme gradient boosting algorithm to generate a model that includes multiple decision trees. In at least some embodiments, individual subsets of the training dataset may be used to train the model ultimately selected as the prediction ML model. For example, a first training dataset subset may be used to train a plurality of models, a second training dataset subset may be used for selecting the prediction ML model from the plurality of models, and a third training dataset subset for testing the model selected as the prediction ML model. The Model Provider may fine-tune the prediction ML model to one or more performance metrics, or other metrics. For example, during fine-tuning of the prediction ML model the Model Provider may vary the number of maximum decision trees, the percentage of the EHR training dataset to train successive decision trees, the learning rate of the model, etc. In at least some embodiments, the performance metric is an AUROC. Features and/or variables that may be indicative of a risk of developing EAC and/or GCA may be discovered during training. FIG. 3A depicts an exemplary AUROC 300 associated with discrimination of the prediction ML model specific to EAC or GCA for a final testing data set, according to some embodiments. FIG. 3B depicts an exemplary bar graph 350 ranking the importance of variables associated with EAC and GCA in terms of the mean Shapley Additive Explanations, according to some embodiments. The Shapley Additive Explanations (SHAPs) measures how much a feature affects the predicted risk on the log-odds scale. FIG. 3C depicts an exemplary bar graph 360 ranking the importance of variables associated with esophageal adenocarcinoma and gastric cardia adenocarcinoma in terms of the proportion of gain in information attributed to each group of features. Once trained, the prediction ML model may generate a prediction of the risk of EAC and/or GCA based upon receiving the EHR dataset of a patient.

The Model Provider may provide the trained prediction ML model to one or more entities such as education institutions, researchers, pharmaceutical companies, healthcare providers, etc. For instance, the Model Provider may enter into an agreement to transfer and/or provide access to the prediction ML model. In one example, the Service Provider provides a computing device (e.g., the server 105, the computing device 115) of a healthcare provider access to the prediction ML model residing on the Model Provider server via a network (e.g., the network 110). In another example, the healthcare provider receives the prediction ML model from the Model Provider for local storage and operation of the prediction ML model via a server of the healthcare provider.

The healthcare provider may apply the prediction ML model to the EHRs datasets of its patients (e.g., hundreds of patients) to determine which patients may be at risk of developing EAC or GCA. For example, a server of the healthcare provider (e.g., the server 105, the computing device 115 may retrieve the EHR datasets of its patients from multiple patient record databases (e.g., the EHR database 160). The healthcare provider may apply the prediction ML model to each patient's EHRs (e.g., via the ML module 142, ML engine 210) and receive an associate prediction of EAC or GCA. The prediction may also include, and/or be used for determining, a patient treatment protocol such as a recommendation for a cancer screening. The prediction may be provided at the server, provided to a computing device of the patient associated with the prediction, a computing device of the patient's healthcare provider, etc. The prediction may be used to determine a patient treatment protocol (e.g., laboratory tests, subsequent check-ups, etc.). FIG. 3D depicts an exemplary display 370 of a computing device including predictions of EAC and/or GCA for patients of the healthcare provider, according to some embodiments. For the patient with a prediction of a higher risk of EAC, the prediction may also include a recommendation for a cancer screening or some other patient treatment protocol. For a patient with a prediction of no/low risk of EAC, there may be no recommendation for a cancer screening.

### Exemplary Computer-Implemented Method for Predicting Esophageal Adenocarcinoma and Gastric Cardia Adenocarcinoma Using Machine Learning

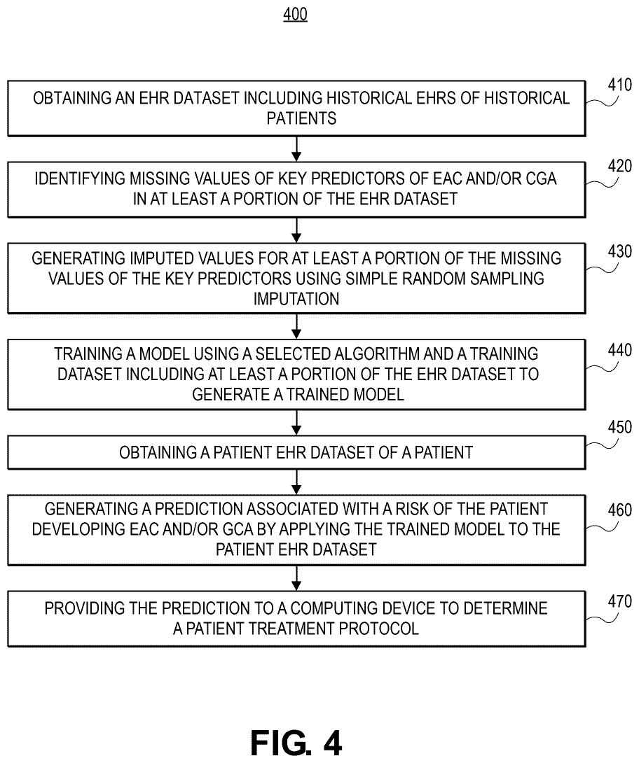

FIG. 4 is a flow diagram depicting an exemplary computer-implemented method 400 for predicting EAC and GCA using machine learning, according to some embodiments. In general, the computer-implemented method 400 may be performed by one or more the devices (e.g., the server 105, the computing device 115), models (e.g., the prediction model 136, the prediction ML model 222), and/or other components of the computing environment 100. One or more steps of the computer-implemented method 400 may be implemented as a set of instructions stored on a computer-readable memory (e.g., the memory 124) and executable by one or more processors (e.g., the processor 120).

The computer-implemented method 400 may include obtaining (e.g., from the EHR database 160) an EHR dataset including historical EHRs of a plurality of historical patients (block 410). The EHR dataset may indicate for the plurality of historical patients one or more of: sex, race, weight, body mass index (BMI), smoking status, Agent Orange exposure, International Classification of Diseases (ICD) codes, prescriptions, or laboratory results.

The computer-implemented method 400 may include identifying missing values of key predictors of EAC and/or GCA in at least a portion of the EHR dataset (block 420). The key predictors may include smoking status, BMI, GERD, or other suitable key predictors.

The computer-implemented method 400 may include generating imputed values for at least a portion of the missing values of the key predictors using simple random sampling imputation (block 430). In at least some embodiments, the computer-implemented method 400 may include generating values of the EHR dataset using simple random sampling imputation for at least a portion of missing values of key predictors of EAC and/or GCA.

The computer-implemented method 400 may include training a model using a selected algorithm and a training dataset to generate a trained model (block 440). The training dataset may include at least a portion of the EHR dataset. The EHR data may include EHR data from at least 1 year before a cancer diagnosis of the historical patients, EHR data of historical patients between ages of 18 and 90 at a time of the cancer diagnosis, or EHR data of the historical patients having at least 2 years of EHR data in a respective historical patient EHR dataset. The selected algorithm may include an extreme gradient boosting algorithm. The trained model may include multiple decision trees.

The training may include tuning (a) a maximum decision tree depth parameter, (b) a number of decision trees, (c) a percentage of the training dataset to train successive decision trees, and (d) a learning rate of the model. In at least some embodiments, (a) the maximum decision tree depth parameter is 6 per decision tree, (ii) the number of the decision trees is 1,000; (iii) the percentage of the training dataset used to train successive decision trees is 50%, and (iv) the learning rate of the model is 0.05. The tuning may achieve at least a threshold value (e.g., the maximum value achieved during model tuning, a value indicating better performance than another model for predicting EAC and/or GCA) of an AUROC associated with a true positive rate and a false positive rate of the model.

The computer-implemented method 400 may include obtaining a patient EHR dataset of a patient (block 450). The patient EHR may be obtained from the computing device 115 (e.g., the computing device of the healthcare provider of the patient), from the EHR database 160, and/or from any other suitable source.

The computer-implemented method 400 may include generating a prediction associated with a risk of the patient developing EAC and/or GCA by applying the trained model to the patient EHR dataset (block 460). In at least some embodiments of the computer-implemented method 400, the prediction may include a metric associated with the risk of the patient developing EAC and/or GCA. If the metric exceeds a threshold, the prediction and/or the patient treatment protocol may include a recommendation for a cancer screening. The metric may be a first metric (e.g., a prediction of a 0.019% chance of developing EAC or GCA) and the recommendation may be for a cancer screening including an upper endoscopy. The metric may be a second metric (e.g., a prediction of a 0.016% chance of developing EAC or GCA) and the recommendation may be for a cancer screening including a non-endoscopic tissue sampling.

The computer-implemented method 400 may include providing the prediction to a computing device to determine a patient treatment protocol (block 470). The computing device 115 may be of the patient, the patient's healthcare provider, and/or other suitable computing device. The patient treatment protocol may include no treatment if the risk is low, a specific type of cancer screening when there is an adequate risk, laboratory test (e.g., bloodwork values), and/or any other suitable patient treatment protocol.

In at least some embodiments, the computer-implemented method 400 may include applying at least one label to at least a portion of the historical EHRs. The labels may indicate (i) a prescription for a proton pump inhibitor, (ii) a prescription for a histamine type 2 receptor antagonists, (iii) a complete blood count value, (iv) a comprehensive metabolic profile, (v) a cholesterol panel, (vi) a hemoglobin A1c value, (vii) a c-reactive protein value, or (viii) an ICD code associated one or more of: (a) heartburn, (b) reflux, or (c) a Charlson comorbidity score component not associated with cancer or metastatic cancer.

In at least some embodiments, the computer-implemented method 400 may include generating, from the EHR dataset, prescription longitudinal summary data including one or more of: a maximum value, a largest increase value, or a total variation value of cumulative daily dosages and average daily dosages of omeprazole and ranitidine equivalents, wherein the prescription longitudinal summary data is used to train the model.

In at least some embodiments, the computer-implemented method 400 may include generating, from the EHR dataset, laboratory longitudinal summary data including one or more of: a maximum value, a minimum value, a mean value, a largest increase value, a largest decrease value, or a total variation value of laboratory data included in the EHR dataset, wherein the laboratory longitudinal summary data is used to train the model.

In at least some embodiments, the computer-implemented method 400 may include generating, from the training dataset, a first dataset for training a plurality of models, a second dataset for selecting the model from the plurality of models, and a third dataset for testing the model.

It should be understood that not all blocks of the exemplary flow diagram of FIG. 4 are required to be performed. Additionally, the computer-implemented method 400 may include fewer, additional, and/or other steps than those depicted in FIG. 4.

### Additional Considerations

With the foregoing, users whose data is being collected and/or utilized may first opt-in. After a user provides affirmative consent, data may be collected from the user's device (e.g., a mobile computing device). In other embodiments, deployment and use of ML models at a client or user device may have the benefit of removing any concerns of privacy or anonymity, by removing the need to send any personal or private data to a remote server.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S.C. § 112 (f) unless traditional means-plus-function language is expressly recited, such as “means for” or “step for” language being explicitly recited in the claim(s). The systems and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment”, “in one aspect” and/or the like in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory product to which it is communicatively coupled. A further hardware module may then, at a later time, access the memory product to retrieve and process the stored output. Hardware modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a building environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Upon reading this disclosure, those of skill in the art will appreciate still additional alternative structural and functional designs for the method and systems described herein through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

Thus, many modifications and variations may be made in the techniques, methods, and structures described and illustrated herein without departing from the spirit and scope of the present claims. Accordingly, it should be understood that the methods and apparatus described herein are illustrative only and are not limiting upon the scope of the claims.

What is claimed:

1. A computer-implemented method for predicting esophageal adenocarcinoma (EAC) and gastric cardia adenocarcinoma (GCA) using machine learning, the computer-implemented method comprising:
obtaining, by one or more processors, an electronic health record (EHR) dataset including historical EHRs of historical patients;
identifying, by the one or more processors, missing values of key predictors of EAC and/or GCA in at least a portion of the EHR dataset;
generating, by the one or more processors, imputed values for at least a portion of the missing values of the key predictors using simple random sampling imputation;
training, by the one or more processors, a model using a selected algorithm and a training dataset including at least a portion of the EHR dataset to generate a trained model, wherein:
(i) the selected algorithm includes an extreme gradient boosting algorithm,
(ii) the trained model includes multiple decision trees, and
(iii) the training includes tuning (a) a maximum decision tree depth parameter, (b) a number of decision trees, (c) a percentage of the training dataset to train successive decision trees, and (d) a learning rate of the model, wherein the tuning achieves a greatest value of an area under a receiver operating characteristic curve associated with true positive rates and false positive rates of the model;
obtaining, by the one or more processors, a patient EHR dataset of a patient;
generating, by the one or more processors, a prediction associated with a risk of the patient developing EAC and/or GCA by applying the trained model to the patient EHR dataset; and
providing, by the one or more processors, the prediction to a computing device to determine a patient treatment protocol specific to the patient based at least upon the prediction associated with the risk of the patient developing EAC and/or GCA.

2. The computer-implemented method of claim 1, wherein the EHR dataset indicates for the historical patients one or more of: sex, race, weight, body mass index (BMI), smoking status, Agent Orange exposure, International Classification of Diseases (ICD) codes, prescriptions, or laboratory results.

3. The computer-implemented method of claim 1, wherein the key predictors include one or more of: smoking status, body mass index, or gastroesophageal reflux disease (GERD).

4. The computer-implemented method of claim 1, further comprising:
applying, by the one or more processors, at least one label to at least a portion of the historical EHRs, the at least one label indicating one or more of: (i) a prescription for a proton pump inhibitor, (ii) a prescription for a histamine type 2 receptor antagonists, (iii) a complete blood count value, (iv) a comprehensive metabolic profile, (v) a cholesterol panel, (vi) a hemoglobin A1c value, (vii) a c-reactive protein value, or (viii) an ICD code associated one or more of: (a) heartburn, (b) reflux, or (c) a Charlson comorbidity score component not associated with cancer or metastatic cancer.

5. The computer-implemented method of claim 1, further comprising:
generating, by the one or more processors from the EHR dataset, prescription longitudinal summary data including one or more of: a maximum value, a largest increase value, or a total variation value of cumulative daily dosages and average daily dosages of omeprazole and ranitidine equivalents, wherein the prescription longitudinal summary data is used to train the model.

6. The computer-implemented method of claim 1, further comprising:
generating, by the one or more processors from the EHR dataset, laboratory longitudinal summary data including one or more of: a maximum value, a minimum value, a mean value, a largest increase value, a largest decrease value, or a total variation value of laboratory data included in the EHR dataset, wherein the laboratory longitudinal summary data is used to train the model.

7. The computer-implemented method of claim 1, wherein the EHR dataset includes one or more of: EHR data from at least 1 year before a cancer diagnosis of the historical patients, EHR data of the historical patients between ages of 18 and 90 at a time of the cancer diagnosis, or EHR data of the historical patients having at least 2 years of EHR data in a respective historical patient EHR dataset.

8. The computer-implemented method of claim 1, wherein one or more of:
(i) the maximum decision tree depth parameter is 6 per decision tree;
(ii) the number of the decision trees is 1,000;
(iii) the percentage of the training dataset to train the successive decision trees is 50%; or
(iv) the learning rate of the model is 0.05.

9. The computer-implemented method of claim 1, further comprising:
generating, by the one or more processors from the training dataset, a first dataset for training a plurality of models, a second dataset for selecting the model from the plurality of models, and a third dataset for testing the model.

10. The computer-implemented method of claim 1, wherein:
the prediction includes a metric associated with the risk; and
based upon the metric exceeding a threshold, the patient treatment protocol includes a recommendation for a cancer screening.

11. The computer-implemented method of claim 10, wherein:
the metric is a first metric and the cancer screening includes an upper endoscopy; or
the metric is a second metric and the cancer screening includes a non-endoscopic tissue sampling.

12. A system for predicting esophageal adenocarcinoma (EAC) and gastric cardia adenocarcinoma (GCA) using machine learning, the system comprising:
one or more processors; and
one or more non-transitory memories storing processor-executable instructions that, when executed by the one or more processors, cause the system to:
obtain an electronic health record (EHR) dataset including historical EHRs of historical patients;
identify missing values of key predictors of EAC and/or GCA in at least a portion of the EHR dataset;
generate imputed values for at least a portion of the missing values of the key predictors using simple random sampling imputation;
train a model using a selected algorithm and a training dataset including at least a portion of the EHR dataset to generate a trained model, wherein:
(i) the selected algorithm includes an extreme gradient boosting algorithm,
(ii) the trained model includes multiple decision trees, and
(iii) to train the model includes tuning (a) a maximum decision tree depth parameter, (b) a number of decision trees, (c) a percentage of the training dataset to train successive decision trees, and (d) a learning rate of the model, wherein the tuning achieves a greatest value of an area under a receiver operating characteristic curve associated with true positive rates and false positive rates of the model;
obtain a patient EHR dataset of a patient;
generate a prediction associated with a risk of the patient developing EAC and/or GCA by applying the trained model to the patient EHR dataset; and
provide the prediction to a computing device to determine a patient treatment protocol specific to the patient based at least upon the prediction associated with the risk of the patient developing EAC and/or GCA.

13. The system of claim 12, wherein the EHR dataset indicates for the historical patients one or more of: sex, race, weight, body mass index (BMI), smoking status, Agent Orange exposure, International Classification of Diseases (ICD) codes, prescriptions, or laboratory results.

14. The system of claim 12, wherein the key predictors include one or more of: smoking status, body mass index, or gastroesophageal reflux disease (GERD).

15. The system of claim 12, further comprising instructions that, when executed by the one or more processors, cause the system to:
apply at least one label to at least a portion of the historical EHRs, the at least one label indicating one or more of: (i) a prescription for a proton pump inhibitor, (ii) a prescription for a histamine type 2 receptor antagonists, (iii) a complete blood count value, (iv) a comprehensive metabolic profile, (v) a cholesterol panel, (vi) a hemoglobin A1c value, (vii) a c-reactive protein value, or (viii) an ICD code associated one or more of: (a) heartburn, (b) reflux, or (c) a Charlson comorbidity score component not associated with cancer or metastatic cancer.

16. The system of claim 12, further comprising instructions that, when executed by the one or more processors, cause the system to generate, from the EHR dataset, one or more of:
prescription longitudinal summary data including one or more of: a maximum value, a largest increase value, or a total variation value of cumulative daily dosages and average daily dosages of omeprazole and ranitidine equivalents, wherein the prescription longitudinal summary data is used to train the model; or
laboratory longitudinal summary data including one or more of: a maximum value, a minimum value, a mean value, a largest increase value, a largest decrease value, or a total variation value of laboratory data included in the EHR dataset, wherein the laboratory longitudinal summary data is used to train the model.

17. The system of claim 12, wherein one or more of:
(i) the maximum decision tree depth parameter is 6 per decision tree;
(ii) the number of the decision trees is 1,000;
(iii) the percentage of the training dataset to train the successive decision trees is 50%; or
(iv) the learning rate of the model is 0.05.

18. The system of claim 12, wherein:
the prediction includes a metric associated with the risk; and
based upon the metric exceeding a threshold, the patient treatment protocol includes a recommendation for a cancer screening.

19. The system of claim 18, wherein:
the metric is a first metric and the cancer screening includes an upper endoscopy; or
the metric is a second metric and the cancer screening includes a non-endoscopic tissue sampling.

20. A non-transitory computer readable medium having processor-executable instruction stored thereon that, when executed by one or more processors, cause the one or more processors to at least:
obtain an electronic health record (EHR) dataset including historical EHRs of historical patients;
identify missing values of key predictors of esophageal adenocarcinoma (EAC) and/or gastric cardia adenocarcinoma (GCA) in at least a portion of the EHR dataset;
generate imputed values for at least a portion of the missing values of the key predictors using simple random sampling imputation;
train a model using a selected algorithm and a training dataset including at least a portion of the EHR dataset to generate a trained model, wherein:
(i) the selected algorithm includes an extreme gradient boosting algorithm,
(ii) the trained model includes multiple decision trees, and
(iii) to train the model includes tuning (a) a maximum decision tree depth parameter, (b) a number of decision trees, (c) a percentage of the training dataset to train successive decision trees, and (d) a learning rate of the model, wherein the tuning achieves a greatest value of an area under a receiver operating characteristic curve associated with true positive rates and false positive rates of the model;
obtain a patient EHR dataset of a patient;
generate a prediction associated with a risk of the patient developing EAC and/or GCA by applying the trained model to the patient EHR dataset; and
provide the prediction to a computing device to determine a patient treatment protocol specific to the patient based at least upon the prediction associated with the risk of the patient developing EAC and/or GCA.
